(19) United States
(12) Patent Application Publication
(10) Pub. No.: US 2021/0085499 A1
(43) Pub. Date: Mar. 25, 2021

(54) TRANSPYLORIC ANCHORING

(71) Applicant: GI Dynamics, Inc., Boston, MA (US)

(72) Inventors: Sean K. HOLMES, East Hampton, CT (US); Barry MAXWELL, Spencer, MA (US); Ezra S. FISHMAN, Cambridge, MA (US)

(21) Appl. No.: 16/876,724

(22) Filed: May 18, 2020

Related U.S. Application Data
(63) Continuation of application No. 15/881,875, filed on Jan. 29, 2018, now abandoned.
Application No. 15/881,875 is a continuation of application No. 14/276,697, filed on May 13, 2014, now abandoned.
Application No. 14/276,697 is a division of application No. 12/787,531, filed on May 26, 2010, now abandoned.
(60) Provisional application No. 61/217,318, filed on May 29, 2009.

(51) Int. Cl. A61F 5/00; A61B 17/11

(52) U.S. Cl. CPC A61F 5/0076; A61B 17/11; A61B 2017/00849; A61F 5/0079; A61B 17/1114

(57) ABSTRACT

Gastrointestinal implants can be used to secure thin-walled sleeves, restrictor plates, and other devices within the gastrointestinal tract. An example implant includes three elements: a stomach anchor to resist distally oriented forces; a duodenal anchor to resist proximally oriented forces; and a connector element to keep the stomach anchor fixed relative to the stomach anchor. The implant is inserted into the gastrointestinal tract with a delivery device that holds the implant in a compressed state for minimally invasive delivery until the implant is positioned properly. Upon releasing from the delivery device, the implant expands to a relaxed state across the pylorus, allowing prongs that extending outward from the stomach and duodenal anchors to engage tissue in the gastrointestinal tract. The deployed implant may also include a thin-walled sleeve that extends into the intestine from the stomach anchor, duodenal anchor, or connector element.

### RELATED APPLICATIONS

This application is a continuation of U.S. application Ser. No. 15/881,875, filed Jan. 29, 2018, which is a continuation of U.S. application Ser. No. 14/276,697, filed May 13, 2014, which is a divisional of U.S. application Ser. No. 12/787,531, filed May 26, 2010, which claims the benefit of U.S. Provisional Application No. 61/217,318, filed on May 29, 2009. The entire teachings of the above applications are incorporated herein by reference.

### BACKGROUND OF THE INVENTION

Obesity is associated with a wide variety of health problems, including Type 2 diabetes, hypertension, coronary artery disease, hypercholesteremia, sleep apnea, and pulmonary hypertension. It also exerts an enormous strain on the body that affects the organs, the nervous system, and the circulatory systems. Obesity rates have been rising for years in the United States, causing corresponding increases in healthcare expenditures.

Curing obesity has so far vexed the best efforts of medical science. Dieting is not an adequate long-term solution for most people, especially those with body-mass indexes of over 30. Stomach stapling, or gastroplasty, reduces the size of the stomach, leading to reduced appetite and weight loss, but eventually the stomach stretches. Roux-en-Y gastric bypass reduces the size of the stomach and the length of the intestine, and leads to both weight loss and alleviation of the Type 2 diabetes common to obese patients. Although gastric bypass appears to provide a more permanent solution than gastroplasty, complication rates associated with gastric bypass are between 2% and 6%, with mortality rates of about 0.5-1.5%.

Endoscopically delivered gastrointestinal implants, such as those described in commonly assigned U.S. Pat. Nos. 7,025,791 and 7,608,114 to Levine et al., incorporated herein by reference in their entireties, provide the benefits of gastric bypass without the hazards of surgery. For example, an implant may include thin-walled, floppy sleeves that are secured in the stomach or intestine with a collapsible anchor. The sleeve extends into the intestine and channels partially digested food, or chyme, from the stomach through the intestine in a manner that may cause weight loss and improve diabetes symptoms. The sleeve and anchor can be removed endoscopically when treatment is over or if the patient desires.

### SUMMARY

A gastrointestinal implant device may include a collapsible stomach anchor and a collapsible duodenal anchor coupled to each other by a radially collapsible coupling member, where the device can be secured across the pylorus. The stomach and duodenal anchors have vertices that define first and second planes, respectively, that are maintained at a substantially constant angle with respect to each other by the coupling member. For example, the coupling member may hold the first and second planes substantially parallel to each other. The example implant device may include an unsupported, thin-walled sleeve that is configured for deployment within the intestine and coupled to the stomach anchor, duodenal anchor, and/or coupling member. The stomach anchor, duodenal anchor, and/or coupling member may also be at least partially covered in a fluoropolymer such that they form a seal that channels chyme (partially digested food) from the stomach through the sleeve.

An example implant device and its components can vary in size depending on whether or not the device is in a relaxed state or a compressed state. When in a relaxed state, an example stomach anchor defines a circle whose diameter is greater than about 60 millimeters. Similarly, a relaxed duodenal anchor can define a circle whose diameter is greater than about 40 millimeters. The diameter of the coupling member may be within a range of from about 10 millimeters to about 25 millimeters, and the coupling member may be within a range of about 30 millimeters to about 60 millimeters in length, e.g., about 40 millimeters long. The example device may be made of single wire, or, alternatively, the stomach anchor, duodenal anchor, and coupling member can be formed of different wires, such as nickel titanium (nitinol) wire with a diameter of about 0.016 inches to about 0.025 inches.

The stomach and duodenal anchors may comprise, respectively, stomach and duodenal prongs that extend outwards from the vertices to secure the implant device across the pylorus. When in a relaxed state, the stomach prongs form a first angle from the first plane, and the duodenal prongs form a second angle with the second plane. Each anchor may include two to six prongs, each of which may be between about 10 millimeters long and about 40 millimeters long; typically, though not necessarily, the stomach prongs are longer than the duodenal prongs. The stomach and duodenal prongs can be arranged in first and second star-shaped configurations, respectively, when viewed axially, and may be arranged so that the first and second star-shaped configurations are arranged in an alternating fashion.

Each prong may include a crown adapted to engage tissue in the gastrointestinal tract, such as in the lower stomach, the pylorus, or the duodenum. The crowns of the stomach and duodenal anchors can define first and second circles whose diameters are greater than about 60 millimeters and about 40 millimeters, respectively, in a relaxed state. Each crown can have a radius of curvature of about 0.1 inch to about 0.4 inch.

Gastrointestinal implant devices can be deployed in the gastrointestinal tract with a delivery device that maintains the stomach and duodenal anchors in respective collapsed states during insertion. The anchors can be configured to self-expand to respective relaxed states when released from the delivery device into the gastrointestinal tract. The stomach and duodenal anchors may expand from their respective collapsed states to their respective relaxed states in a variety of different ways. For example, at least one of the anchors may “spring open”—that is, it may form an acute angle with the coupling member in its respective collapsed state and an angle greater than the acute angle with the coupling member in its respective relaxed state. Alternatively, at least one of the anchors may “spring shut” from an obtuse angle formed with the coupling member in its respective collapsed state to an angle smaller than the obtuse angle in its respective relaxed state.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIGS. 1A and 1B show schematic illustrations of a transpyloric anchor before and after insertion.

FIGS. 2A-2D show schematic illustrations of a transpyloric anchor.

FIGS. 3A and 3B show perspective and plan views, respectively, of a transpyloric anchor.

FIGS. 4A and 4B show perspective and plan views, respectively, of an alternative transpyloric anchor.

FIG. 5 shows a perspective view of an obesity/diabetes treatment device with another alternative transpyloric anchor.

FIG. 6 shows schematic elevation views of transpyloric anchors.

FIGS. 7A and 7B show elevation and plan views of a transpyloric anchor used to secure a restrictor plate across the pylorus.

### DETAILED DESCRIPTION

A description of example embodiments of the invention follows.

Transpyloric anchors are disclosed as alternatives to anchors provided in implants disclosed in U.S. Pat. Nos. 7,025,791; 7,608,114; 7,476,256; U.S. patent application Ser. No. 11/330,705; and U.S. patent application Ser. No. 11/827,674, all of which are incorporated herein by reference in their entireties.

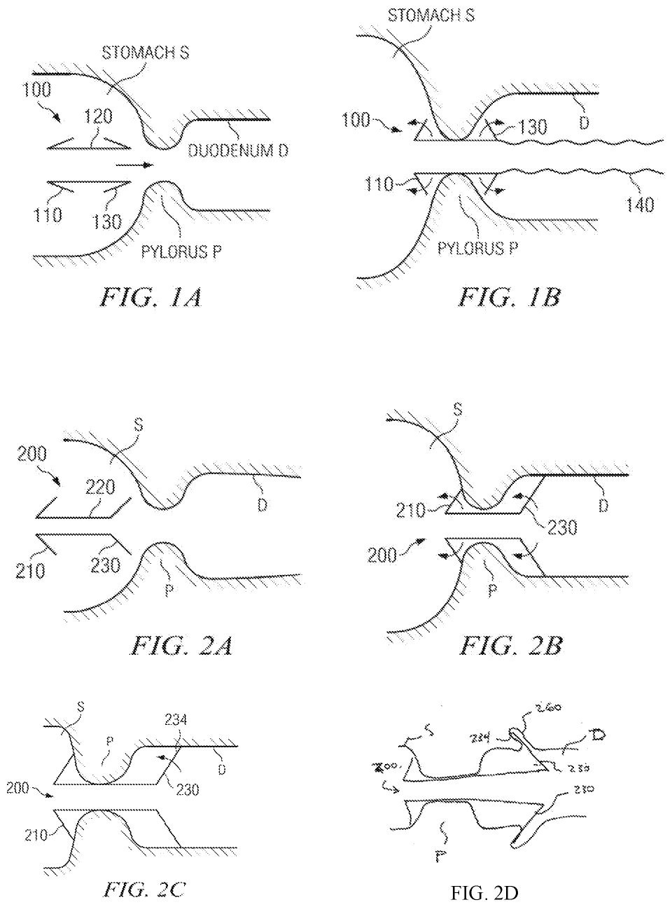

FIGS. 1A and 1B show schematic illustrations of a transpyloric anchor 100 capable of securing a sleeve 140 for treating obesity and/or type-2 diabetes in the intestine of a patient. The sleeve 140, which may extend into the duodenum D and jejunum J for about 60 cm or more, creates a bypass of the proximal intestine. The anchor 100 secures the proximal end of the sleeve 140 in the pylorus P, which connects the stomach S to the duodenum D, and forms a seal between either the stomach S or the pylorus P and the duodenum D. (FIG. 1B shows the anchor 100 forming a seal between the pylorus P and the duodenum D.) The seal ensures that most of the partially digested food, or chyme, enters the sleeve 140 and thereby avoids contact with the walls of the intestine. Thus, the anchor directs chyme from the stomach through the sleeve 140, which may effect weight loss and the improvement in diabetes symptoms.

FIG. 1A shows the transpyloric anchor 100 in one of its undeployed configurations. A stomach anchor, or proximal member 110, and a duodenal anchor, or distal member 130, are folded towards the center of a connector 120, which is strong enough to withstand pushing, pulling, stretching, and twisting forces on the transpyloric anchor 100. Once the anchor 100 is inserted into the pylorus, the proximal and distal members 110, 130 spring into a deployed configuration in the direction of the arrows shown in FIG. 1B. Once the members 110, 130 are deployed, the tips, or crowns, of the members 110, 130 engage the walls of the stomach S and duodenum D. Forces exerted by the crowns against the stomach S and duodenum D cause the anchor 100 to resist both proximally and distally directed forces, securing the anchor 100 within the pylorus. Thus, the transpyloric anchor 100 secures the sleeve 140 within the duodenum D without the use of barbs. Because the transpyloric anchor 100 does not use barbs, it may be removed simply by pulling proximally with an endoscopic device.

FIGS. 2A-2D show schematic illustrations of an alternative transpyloric anchor 200 and its deployment within the pylorus. As above, the transpyloric anchor 200 is inserted into the pylorus with folded proximal and distal members 210 and 230, which are secured to each other with a connector 220. In this embodiment, however, the distal member 230 is folded away from the center of the connector 220, as shown in FIG. 2A. When the transpyloric anchor 200 is inserted into the pylorus P, the distal member 230 deploys by springing back towards the stomach (i.e., in the direction of the arrows), which, in turn, causes crowns 234 to engage the walls of the duodenum D, as shown in FIG. 2B. Because the distal member 230 forms an acute angle with the connector 220 in its relaxed state, the distal member 230 continues to push against the wall of the duodenum D, as shown in FIG. 2C. Over time (e.g., about one week), this spring action causes the crowns 234 to form folds, or plications 260, in the duodenum D that may permanently fix the anchor 200 in position, as shown in FIG. 2D. The proximal member 210 can also be folded away from the center of the connector 220 either in addition or instead of folding the distal member away from the center of the connector 220. The transpyloric anchor 200 may be removed by pulling proximally without inflicting serious or lasting damage to the patient. By forming these plications, the serosal layers of the folded tissues may adhere to each other thereby forming permanent plications or folds in which the anchor may engage. Plications may also be formed solely in the duodenum. By placing the anchor 200 in the duodenum, plications on the distal end of the anchor may be formed, enhancing the anchoring of the device. If the distal members are also placed on the proximal end, then plications may form on both the proximal and distal end of the anchor. A method of treatment may comprise securing an anchor in a lumen by causing plications to form in tissue of the lumen over at least part of the anchor. A method of treatment may comprise securing an anchor across a pylorus by causing plications to form in the stomach or duodenum over crowns extending from the anchor.

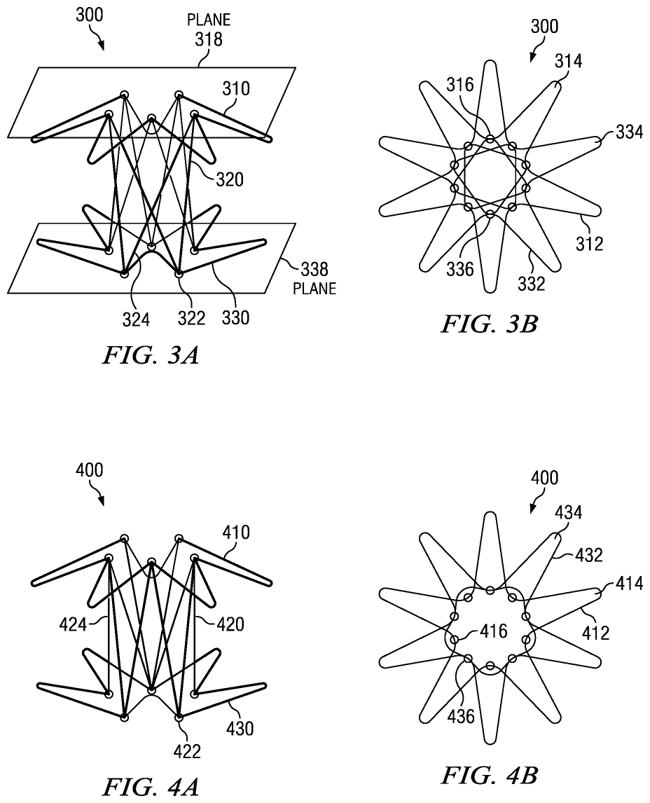

FIGS. 3A and 3B show perspective and plan views, respectively, of a transpyloric anchor 300 capable of securing a sleeve in the intestine of a patient for treating obesity and/or type-2 diabetes. The transpyloric anchor 300 includes a collapsible stomach anchor, or proximal member 310 coupled to a collapsible duodenal anchor, or distal member 330 by a radially collapsible coupling member, or connector 320. The proximal member 310 is formed of a single wire in the shape of star, with stomach prongs 312 that have tips, or crowns 314, that engage the interior of the stomach. The number of stomach prongs 312 varies depending on the size of the device, the location of the seal, and the strength of the wire; typically, there are two to six prongs on the stomach anchor 310. The number of crowns 314 per prong 312 may also vary.

The distal member 330 is also a single wire formed into a star configuration of duodenal prongs 332, each of which has a crown 334. The number of duodenal prongs 332, the number of crowns 334, and rotational orientation of the distal member 330 with respect to the proximal member 310 depends on the wire strength and the location of the seal. For example, the proximal and distal members 310, 330 may be aligned in phase with each other or slightly out of phase with each other such that they press against opposite sides of the tissue separating the proximal and distal members 310, 330. Arranging the proximal and distal members 310, 330 in phase or slightly out of phase with each other improves resistance to forces exerted along the longitudinal axis of the intestine, but may cause erosion of the tissue between the stomach and duodenal prongs 312, 332. Alternatively, the proximal and distal members 310, 330 may be aligned out of phase with each other, as shown in FIG. 3B, to prevent the stomach and duodenal prongs 312, 332 from eroding through the stomach and the duodenum.

The stomach and duodenal prongs 312, 332 flare outwards from the proximal and distal members 310, 330 and trace out circular envelopes when viewed along the longitudinal axis of the anchor 300. The envelopes have diameters that are large enough to prevent the anchor 300 from being pulled through the pylorus in either direction. For example, when relaxed, the crowns 314 of the stomach prongs 312 may trace a circle with a diameter greater than about 50 millimeters, or, more preferably, greater than about 60 millimeters, to prevent the anchor 300 from being pulled into the intestine. Similarly, the crowns 334 of the duodenal prongs 332, when in a relaxed state, may trace a circle with a diameter of greater than about 40 millimeters to prevent the anchor from being pulled through the pylorus into the stomach. Each stomach and duodenal prong 312, 332 is preferably between about 10 and about 40 millimeters long, and, more preferably, between about 15 and 30 millimeters long. The stomach and duodenal prongs 312, 332 may bend under loading, changing the shape and size of the envelope traced by the stomach and duodenal prongs 312, 332.

The connector 320 maintains a fixed angle between the proximal anchor 310 and the distal anchor 330. The proximal anchor 310 defines a plane 318 at the connection between the coupling member 320 and the proximal anchor 310. The connection between the distal anchor 330 and the coupling member 320 defines a second plane 338. The coupling member 320 should have sufficient stiffness linearly to maintain a fixed angle between plane 318 and plane 338. Preferably, as shown in FIG. 3A, this angle is zero (0) degrees such that the planes are parallel to each other and perpendicular to the axis of the transpyloric anchor. However, either of the stomach and duodenal anchors 310, 330 may be positioned such that their planes are angled, for example, between about 75 degrees and about 90 degrees with respect to the coupling member 320.

The connector 320 is preferably able to collapse easily and sufficiently enough for the pylorus to function. The radial force required to collapse the connector 320 diameter by 50% should be preferably no greater than about 0.5 lbs. Thus, the connector 320 may be rigid in the longitudinal direction, but radially collapsible. Here, the connector 320 is a single wire that connects the proximal and distal members 310, 330. Loops 322 in the connector 320 hold the inner points of the members 310, 330—that is, the vertices, or junctions 316, 336 between adjacent prongs 312, 332. The wire segments 324 connecting the loops 322 are woven together, allowing the connector 320 to flex without comprising the connection between the proximal and distal members 310, 330.

When the transpyloric anchor 300 is in a relaxed state, the stomach and duodenal prongs 312, 332 flare outwards from the planes 318, 338 defined by the vertices 316, 336 at either end of the coupling member 320. Depending on the configuration, the prongs 312, 332 may form acute or obtuse angles with the long axis of the connector 320. In this example, both the stomach prongs 312 and the duodenal prongs 332 form acute angles with the coupling member 320—i.e., the crowns 314, 334 fold towards the center of the coupling member 320 when uncompressed. Alternatively, the crowns 314, 334 may point away from the coupling member 320 when uncompressed; in some cases, one set of prongs 312, 332 may form an obtuse angle with the coupling member 320 and the other set of prongs 312, 332 may form an acute angle with the coupling member 320.

FIGS. 4A and 4B show perspective and plan views, respectively, of an alternative transpyloric anchor 400. Like the transpyloric anchor 300 shown in FIGS. 3A and 3B, the transpyloric anchor 400 includes proximal and distal members 410, 430, each of which have prongs 412, 432 in star configurations with crowns 414, 434 that engage the walls of the stomach and duodenum, respectively. A connector 420 couples the members 410, 430 to each other using loops 422 and wire segments 424 that connect alternating junctions 416, 436 of the star configurations of the proximal and distal members 410, 430, respectively. Unwoven connectors 420 trade the rigidity of woven connectors (e.g., connector 320 of FIGS. 3A and 3B) for an improved ability to collapse to a smaller diameter.

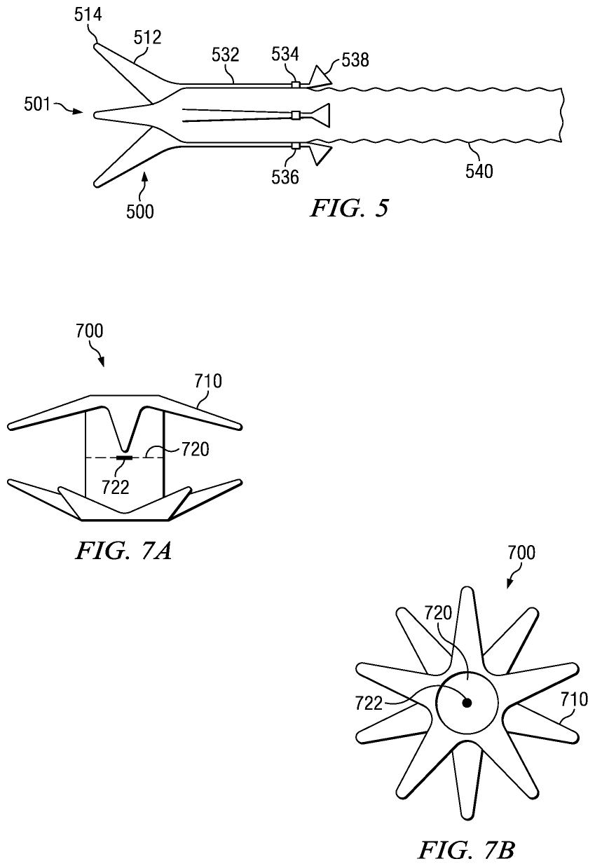

FIG. 5 shows a perspective view of an alternative transpyloric anchor 500 coupled to a sleeve 540 to form an obesity/diabetes treatment device 501. The transpyloric anchor 500 is formed of a single loop of wire in a six-pronged star configuration, with three proximal prongs 512 spaced in an alternating fashion with three distal prongs 532. As shown in FIG. 5, the proximal prongs 512 are shaped into broad wedges, which are covered to close gaps between the anchor 500 and the stomach. Crowns 514 at the ends of the proximal prongs 512 engage the stomach to prevent the anchor 500 from being pulled into the intestine. Spring force exerted by the anchor 500 causes feet 538, which are crimped to crowns 534 with metal bands 536 at the ends of the narrow distal prongs 532, engage the duodenum and/or pylorus. The feet 538 may engage the duodenal wall to fix the anchor 500 into position. The transpyloric anchor 500 may be coated, covered, or wrapped in sheet of material to keep chyme in the sleeve 540, which extends into the intestine. If chyme slips between the sleeve 540 and the intestine, the effectiveness of the anti-obesity/diabetes treatment device may be reduced.

In general, any transpyloric anchor may be coupled to a thin-walled sleeve that is configured to extend into the intestine. The sleeve may be made of a fluoropolymer, such as expanded polytetrafluoroethylene (ePTFE) coated or impregnated with fluorinated ethylene polyethylene (FEP), or any other suitable material, and the transpyloric anchor may be coated, covered, or wrapped in the same material used to form the sleeve. A typical sleeve is floppy and conformable to the wall of the intestine when deployed. It also has a wall thickness of less than about 0.0005 inch to about 0.001 inch and a coefficient of friction of about 0.2 or less. The sleeve and anchor covering can be a single, integrally formed piece. They can also be separate pieces, depending on whether the transpyloric anchor is partially or wholly uncovered, as long as the transpyloric anchor forms a sufficiently good seal between the sleeve and the stomach, pylorus, and/or intestine.

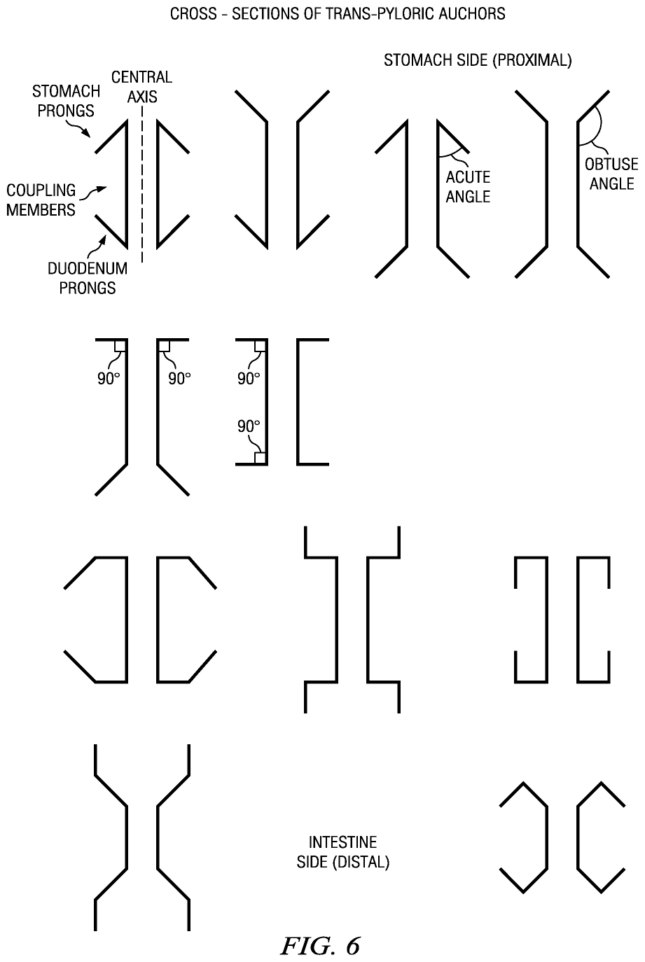

FIG. 6 shows schematic elevations views of transpyloric anchors with a variety of different prong configurations. Prongs may form acute, obtuse, and/or right angles with the connectors in both deployed and undeployed configurations. In general, the prongs extend outwards at an angle from the planes defined by the connections of the proximal and distal anchors and the connecting member. Prongs may also be bent multiple times, as shown in the third and fourth rows of FIG. 6. FIG. 6 is not exhaustive; other combinations of prong configurations are also possible. Proximal prongs typically span diameters of more than about 50 mm to prevent the anchors from being pulled into the intestine. For example, the diameter spanned by the proximal prongs may be about 50 mm or more, and is preferably about 60 mm or more. Similarly, distal prongs usually span diameters of about 40 mm or more to prevent the anchors from being pulled into the stomach. Connector diameters range from about 10 mm to about 25 mm, depending on the location of the seal. Transpyloric anchors with seals on either side of the pylorus may have connectors with smaller diameters. Connectors with larger diameters may press up against the pylorus to tightly close gaps between the anchor and the pylorus.

Transpyloric anchors may be inserted endoscopically in a variety of undeployed configurations. Once inserted, a transpyloric anchor may self-expand across the pylorus, as shown in FIGS. 1 and 2, to secure a sleeve within the duodenum. For example, a gastrointestinal implant device that includes a transpyloric anchor and a sleeve can be inserted into the gastrointestinal tract with the devices and methods disclosed in U.S. Pat. Nos. 7,678,068 and 7,122,058, both of which are incorporated herein by reference in their entireties. The deployed transpyloric anchor and attached sleeve can be removed endoscopically by simply pulling the anchor towards the patient's mouth. To minimize trauma during retrieval, the transpyloric anchor can be pulled out with a hooded retrieval device: first, the retrieval device is used to grasp a drawstring that runs through the transpyloric anchor. Pulling the drawstring collapses the transpyloric anchor. The hood shields the gastrointestinal tract from the collapsed transpyloric anchor as the anchor is withdrawn, as described in U.S. patent application Ser. No. 12/005,049, filed Dec. 20, 2007, and incorporated herein by reference in its entirety.

FIGS. 7A and 7B show elevation and plan views, respectively, of an alternative implant 700 that includes a transplyoric anchor 710 covered in fluoropolymer. The transplyoric anchor 710 is used to secure a restrictor plate 720 within the gastrointestinal tract to treat obesity, as disclosed in U.S. patent application Ser. No. 10/811,293, U.S. patent application Ser. No. 11/330,705, and U.S. patent application Ser. No. 11/827,674, all of which are incorporated herein by reference in their entireties. The restrictor plate 720 has a restricting aperture 722 at its center that retards the outflow of food from the stomach to the intestine. The diameter of the aperture 722 is less than about 10 millimeters, and is preferably in the range of about 3-7 millimeters. The aperture 722 may be elastic and expandable under pressure from material flowing through the anchor and the aperture at elevated physiological pressures; as pressure increases, the apertures opens to greater diameters. The restrictor plate 720 and/or the anchor 710 may also be coupled to a sleeve (not shown) that extends into the intestine.

While this invention has been particularly shown and described with references to example embodiments thereof, it will be understood by those skilled in the art that various changes in form and details may be made therein without departing from the scope of the invention.

1. A method of treatment comprising:
providing a gastrointestinal implant device, the device comprising:
a collapsible stomach anchor having stomach prongs that extend outward in a relaxed state, crowns of the stomach prongs defining a circle with a diameter of about 50 millimeters or more;
a collapsible duodenal anchor having duodenal prongs that extend outward in a relaxed state; and
a radially collapsible coupling member that couples the stomach anchor to the duodenal anchor, the coupling member maintaining the stomach anchor at a substantially constant position with respect to the duodenal anchor; and
securing the device across the pylorus of a patient.

2. The method of claim 1, wherein the coupling member keeps the stomach anchor and the duodenal anchor substantially parallel to each other.

3. The method of claim 1, wherein securing the device includes allowing at least one crown on at least one of the stomach prongs and the duodenal prongs to engage tissue in the gastrointestinal tract.

4. The method of claim 3, wherein allowing the at least one crown to engage tissue comprises causing a plication to form in the stomach or duodenum over the at least one such crown.

5. The method of claim 1, further including:
forming a seal with the gastrointestinal implant device.

6. The method of claim 5, further including:
channeling chyme from the stomach through an unsupported, thin-walled sleeve extending into the intestine from the gastrointestinal implant device.

7. The method of claim 5, further comprising:
restricting a flow of chyme from the stomach into the intestine with a restrictor plate coupled to the gastrointestinal implant device.

8. The method of claim 1, further comprising:
maintaining the stomach and duodenal anchors in respective collapsed states using a delivery device; and
allowing the stomach and duodenal anchors to expand towards respective relaxed states by releasing the stomach and duodenal anchors from the delivery device.

9. The method of claim 8, wherein allowing the duodenal anchor to expand towards a respective relaxed state includes allowing the spring arms to expand away from or towards the coupling member.

10. (canceled)

11. A method of treatment comprising securing a gastrointestinal implant device across a pylorus of a patient, the method comprising:
releasing a collapsible stomach anchor on a stomach side of a pylorus of a patient, the releasing comprising releasing stomach prongs of the collapsible stomach anchor to a relaxed state so that the crowns of the stomach prongs define a circle with a diameter of about 50 millimeters or more; and
releasing a collapsible duodenal anchor on a duodenal side of the pylorus of the patient, the releasing comprising releasing duodenal prongs of the collapsible duodenal anchor to a relaxed state;
wherein a radially collapsible coupling member couples the stomach anchor to the duodenal anchor, the coupling member maintaining the stomach anchor at a substantially constant position with respect to the duodenal anchor.

12. The method of claim 11, wherein the coupling member keeps the stomach anchor and the duodenal anchor substantially parallel to each other.

13. The method of claim 11, wherein securing the device includes allowing at least one crown on at least one of the stomach prongs and the duodenal prongs to engage tissue in the gastrointestinal tract.

14. The method of claim 13, wherein allowing the at least one crown to engage tissue comprises causing a plication to form in the stomach or duodenum over the at least one such crown.

15. The method of claim 11, further including:
forming a seal with the gastrointestinal implant device.

16. The method of claim 15, further including:
channeling chyme from the stomach through an unsupported, thin-walled sleeve extending into the intestine from the gastrointestinal implant device.

17. The method of claim 15, further comprising:
restricting a flow of chyme from the stomach into the intestine with a restrictor plate coupled to the gastrointestinal implant device.

18. The method of claim 11, further comprising:
maintaining the stomach and duodenal anchors in respective collapsed states using a delivery device; and
allowing the stomach and duodenal anchors to expand towards respective relaxed states by releasing the stomach and duodenal anchors from the delivery device.

19. The method of claim 18, wherein allowing the duodenal anchor to expand towards a respective relaxed state includes allowing the spring arms to expand away from or towards the coupling member.

20. (canceled)

21. A method of treatment comprising securing a gastrointestinal implant device across a pylorus of a patient, the method comprising:
releasing a collapsible stomach anchor on a stomach side of a pylorus of a patient, the releasing comprising releasing spring arms of the collapsible stomach anchor to a relaxed state so that the collapsible stomach anchor defines a circle with a diameter of about 50 millimeters or more; and
releasing a collapsible duodenal anchor on a duodenal side of the pylorus of the patient, the releasing comprising releasing spring arms of the collapsible duodenal anchor to a relaxed state;
wherein a radially collapsible coupling member couples the stomach anchor to the duodenal anchor, the coupling member maintaining the stomach anchor at a substantially constant position with respect to the duodenal anchor.

22. The method of claim 21, wherein the coupling member keeps the stomach anchor and the duodenal anchor substantially parallel to each other.

23. The method of claim 21, further including:
forming a seal with the gastrointestinal implant device.

24. The method of claim 23, further including:
channeling chyme from the stomach through an unsupported, thin-walled sleeve extending into the intestine from the gastrointestinal implant device.

25. The method of claim 23, further comprising:
restricting a flow of chyme from the stomach into the intestine with a restrictor plate coupled to the gastrointestinal implant device.

26. The method of claim 21, further comprising:
maintaining the stomach and duodenal anchors in respective collapsed states using a delivery device; and
allowing the stomach and duodenal anchors to expand towards respective relaxed states by releasing the stomach and duodenal anchors from the delivery device.

27. The method of claim 26, wherein allowing the duodenal anchor to expand towards a respective relaxed state includes allowing the spring arms to expand away from or towards the coupling member.

28. (canceled)
